(19) United States
(12) Patent Application Publication
(10) Pub. No.: US 2026/0035640 A1
(43) Pub. Date: Feb. 5, 2026

(54) CLEANING AGENT FOR TONERS, METHOD FOR PRODUCING CLEANING AGENT FOR TONERS, AND TONER COMPOSITION

(71) Applicant: NOF CORPORATION, Tokyo (JP)

(72) Inventors: Takeshi Yoshimura, Amagasaki-shi (JP); Mako Nakamura, Amagasaki-shi (JP)

(73) Assignee: NOF CORPORATION, Tokyo (JP)

(21) Appl. No.: 18/997,638

(22) PCT Filed: Aug. 2, 2023
(86) PCT No.: PCT/JP2023/028209

§ 371 Date: Jan. 22, 2025

(30) Foreign Application Priority Data
Aug. 9, 2022 (JP) 2022-126804

(51) Int. Cl. C11D 9/00; C07C 53/126; C11D 11/04; G03G 9/08

(52) U.S. Cl. CPC C11D 9/002; C07C 53/126; C11D 11/04; G03G 9/08

(57) ABSTRACT

To provide a cleaning agent for toners, which is configured to impart to toners appropriate flowability that enables exertion of excellent cleanability and which is configured to suppress wear of the cleaning blade even in a high temperature and high humidity environment. Another object of the present invention is to provide a method for producing the cleaning agent for toners, and a toner composition comprising the cleaning agent for toners and toner base particles. The cleaning agent for toners is a fatty acid divalent metal salt obtained by double decomposition between a fatty acid-alkali compound salt containing 8 to 24 carbon atoms and a divalent metal salt, wherein the fatty acid divalent metal salt has a water activity value A of from 0.65 to 0.85 at 25° C. and a number average value B of area envelopment degrees of from 0.910 to 0.990.

### TECHNICAL FIELD

The present invention relates to a cleaning agent for use in electrostatic latent image developing toners (hereinafter may be simply referred to as “toners”), a method for producing the cleaning agent, and a toner composition comprising the cleaning agent.

### BACKGROUND ART

A toner for use in electrophotographic image forming devices such as a copying machine and a printer, is composite particles such that the surface of toner base particles, which contain a thermoplastic resin (e.g., a polyester-based resin, a styrene-acrylic resin) serving as a binder resin, a colorant (e.g., a carbon black, a magnetic powder, a pigment), a charge control agent, a wax and so on, is coated with a flowability imparting agent, a cleaning agent and so on.

In conventional electrophotographic image forming devices, a cleaning agent is supplied to the surface of an electrophotographic photoconductor (hereinafter may be simply referred to as “photoconductor”) to decrease a frictional force against a blade for cleaning the photoconductor surface (hereinafter, it may be simply referred to as “cleaning blade”), thereby preventing the toner from passing through the blade and thus suppressing filming, and preventing wear of the photoconductor surface.

As the method for supplying the cleaning agent to the photoconductor surface, examples include, but are not limited to, the following methods: (1) using a cleaning agent application system (an applicator), (2) adding the cleaning agent to the surface layer of the photoconductor, and (3) adding the cleaning agent to coat the toner particles and then supplying the coated toner particles to the photoconductor surface concurrently with development.

As the method for supplying the cleaning agent by (1) using the cleaning agent application system, examples include, but are not limited to, installing a cleaning agent applying device for applying the cleaning agent to the photoconductor surface. This method is advantageous in that the cleaning agent can be evenly supplied to the whole photoconductor surface. However, this method needs a dedicated device, a space for installing the applying device, or the like, and enlargement and complication of the image forming device is inevitable. In addition, there is a problem of uneven application of the cleaning agent, which is caused by a deteriorated coating member, or there is a problem of complicated maintenance such as a need for an additional cleaning agent supplying device.

The method for supplying the cleaning agent by (2) adding the cleaning agent to the surface layer of the photoconductor, has a certain effect of suppressing the wear of the photoconductor surface. However, partial variation in the properties of the photoconductor surface, such as a partial decrease in sensitivity, occurs and results in a problem such as an image defect.

The method for supplying the cleaning agent by (3) adding the cleaning agent to coat the toner particles and then supplying the coated toner particles to the photoconductor surface concurrently with development, is employed in many electrophotographic image forming devices since the method enables a reduction in device size and easy and simple supply of the cleaning agent.

As the cleaning agent used in the method (3), a fatty acid metal salt has been preferably used. Due to its excellent slidability, the use of a fatty acid metal salt for blade cleaning stability and suppression of non-uniform wear (uneven wear) has been studied.

When transporting a cartridge including a toner, it may be exposed to a high temperature environment of 50° C. or more, especially in summer. In recent years, due to global spread of printers, multifunctional printers and so on, they may be used for a long period of time while being exposed to a high temperature and high humidity environment. Accordingly, a fatty acid metal salt is required to have the property of being able to be dispersedly attached to toner particles to impart excellent flowability to the toner particles even in the above-described environments and the property of being able to quickly detach the toner particles from the cleaning blade. Along with the speed up of printers, there is also a need for a fatty acid metal salt which can more quickly detach the toner particles from the cleaning blade.

For example, Patent Document 1 proposes the following technique: fatty acid metal salt particles having a volume average particle diameter of from 3 μm to 15 μm are added as an external additive on toner base particles so that the ratio (B/A) of the number B of the fatty acid metal salt particles observed in a specific field of view to the proportion A (% by weight) of the fatty acid metal salt particles contained in the entire toner is in a specific range. According to Patent Document 1, by the external addition of the fatty acid metal salt particles having the above-described specific particle diameter so as to obtain the specific range (B/A), cleanability is improved; wear of the surface of a photoconductor by the cleaning blade is suppressed; and the electrostatic properties of the toner are stabilized, thereby enabling excellent image formation without image defects.

Patent Document 2 proposes a technique to use fatty acid metal salt particles as an external additive of a toner, which have an iron content of from 0.0008% by mass to 0.01% by mass. According to Patent Document 2, by use of the fatty acid metal salt particles having an iron content in the above range, lateral streaks are less likely to appear in images formed on recording media, even after repeated image-forming operations.

Patent Document 3 proposes a technique to use fatty acid metal salt particles as an external additive of a toner, which have a sulfur element content of from 0.0035% by mass to 0.07% by mass. According to Patent Document 3, by use of the fatty acid metal salt particles having a sulfur element content in the above range, wear of a blade for cleaning an image carrier can be reduced.

### CITATION LIST

### Patent Documents

Patent Document 1: Japanese Patent Application Laid-Open (JP-A) No. 2000-089502
Patent Document 2: JP-A No. 2017-125928
Patent Document 3: JP-A No. 2016-218409

### SUMMARY OF INVENTION

### Technical Problem

However, even with the techniques described in Patent Documents 1 to 3, it is still difficult to suppress wear of the cleaning blade in a high temperature and high humidity environment.

The present invention was achieved in light of the above circumstances. An object of the present invention is to provide a cleaning agent for toners, which is configured to impart to toners appropriate flowability that enables exertion of excellent cleanability and which is configured to suppress wear of the cleaning blade even in a high temperature and high humidity environment. Another object of the present invention is to provide a method for producing the cleaning agent for toners, and a toner composition comprising the cleaning agent for toners.

### Solution to Problem

As a result of intensive studies with the aim of achieving the above objects, the present inventors found that excellent performance can be exerted when a fatty acid divalent metal salt having a specific water activity value and a specific area envelopment degree is used as a cleaning agent for toners, and they achieved the present invention.

The cleaning agent for toners according to the present invention is a fatty acid divalent metal salt obtained by double decomposition between a fatty acid-alkali compound salt, which is obtained by reaction of a fatty acid containing 8 to 24 carbon atoms with a monovalent alkali compound, and a divalent metal salt,
wherein the fatty acid divalent metal salt has a water activity value A of from 0.65 to 0.85 at 25° C., which is calculated by the following formula (1), and a number average value B of area envelopment degrees of from 0.910 to 0.990, each of which is defined as a ratio of a projected area to an area inside an envelope (the projected area/the area inside the envelope) for a projected image, and the area envelopment degrees are those of projected images of fatty acid divalent metal salt having an equivalent circle diameter in a range of from a cumulative 10% diameter to a cumulative 90% diameter on a volumetric basis in the fatty acid divalent metal salt:

$$A = P / {P}_{0} \qquad Formula ( 1 )$$

P: water vapor pressure (Pa) at 25° C. inside a closed container containing the fatty acid divalent metal salt
P₀: vapor pressure (Pa) of water at 25° C.

A volume-based median diameter (D50) of the fatty acid divalent metal salt that is the cleaning agent for toners according to the present invention, which is measured by a laser light diffraction scattering method, is preferably from 0.3 μm to 5.0 μm.

A divalent metal contained in the fatty acid divalent metal salt, which is the cleaning agent for toners according to the present invention, is preferably at least one selected from the group consisting of zinc, calcium and magnesium.

The method for producing the cleaning agent for toners according to the present invention is a method for producing the fatty acid divalent metal salt, which is the cleaning agent for toners according to the present invention,
the method comprising:
preparing a slurry of a fatty acid divalent metal salt by a double decomposition method in which, in an aqueous solution, a divalent metal salt is reacted with a fatty acid-alkali compound salt obtained by reaction of a fatty acid containing 8 to 24 carbon atoms with a monovalent alkali compound,
obtaining a water-containing cake of the fatty acid divalent metal salt from the slurry, and
drying and pulverizing the water-containing cake by hot-air drying of the water-containing cake at a temperature which is equal to or more than a melting point of the fatty acid divalent metal salt and is 200° C. or less.

The toner composition according to the present invention is a toner composition comprising the cleaning agent for toners according to the present invention and toner base particles,
wherein a content of the cleaning agent for toners is from 0.01 parts by mass to 5 parts by mass, with respect to 100 parts by mass of the toner base particles.

### Advantageous Effects of Invention

Compared to a fatty acid metal salt such that the water activity value A at 25° C. is outside the range of from 0.65 to 0.85 or the number average value B of the area envelopment degrees is outside the range of from 0.910 to 0.990, the fatty acid divalent metal salt that is the cleaning agent for toners according to the present invention can impart to toners appropriate flowability that enables exertion of excellent cleanability, and it can suppress wear of the cleaning blade even in a high temperature and high humidity environment.

### DESCRIPTION OF EMBODIMENTS

Hereinafter, the embodiments of the present invention will be described. The cleaning agent for toners and the toner composition according to the present invention will be described in this order.

In the present invention, a numerical range defined by use of “to” includes the numerical values at both ends of “to” (i.e., the upper and lower limit values). For example, “10 to 30” indicates a range of 10 or more and 30 or less.

### (1) Cleaning Agent for Toners

The cleaning agent for toners according to the present invention is a cleaning agent for toners, which is a fatty acid divalent metal salt obtained by double decomposition between a fatty acid-alkali compound salt, which is obtained by reaction of a fatty acid containing 8 to 24 carbon atoms with a monovalent alkali compound, and a divalent metal salt,
wherein the fatty acid divalent metal salt has a water activity value A of from 0.65 to 0.85 at 25° C., which is calculated by the following formula (1), and a number average value B of area envelopment degrees of from 0.910 to 0.990, each of which is defined as a ratio of a projected area to an area inside an envelope (the projected area/the area inside the envelope) for a projected image, and the area envelopment degrees are those of projected images of fatty acid divalent metal salt having an equivalent circle diameter in a range of from a cumulative 10% diameter to a cumulative 90% diameter on a volumetric basis in the fatty acid divalent metal salt:

$$A = P / {P}_{0} \qquad Formula ( 1 )$$

P: water vapor pressure (Pa) at 25° C. inside a closed container containing the fatty acid divalent metal salt
P₀: vapor pressure (Pa) of water at 25° C.

Adding a fatty acid metal salt (e.g., zinc stearate) on toner particles as a cleaning agent, is a conventionally known technique. The fatty acid metal salt is released from the toner particles; the released fatty acid metal salt is transferred to the surface of a photoconductor; and the fatty acid metal salt on the photoconductor surface functions as a lubricant between the photoconductor and the cleaning blade, thereby suppressing wear of the cleaning blade. However, when images are formed with the toner on which the fatty acid metal salt is added, the degree of wear progression of the cleaning blade may be varied in an axial direction (the axial direction of the photoconductor, i.e., the direction perpendicular to the driving direction of the driven photoconductor) or the progression of the wear of the entire cleaning blade may be faster than expected. The reason is presumed as follows.

The fatty acid metal salt is a compound obtained by reaction in water. Considering its constitutional materials, the fatty acid metal salt contains a certain amount of water. Due to containing a certain amount of water, the fatty acid metal salt is likely to be positively charged by friction inside a developing device. Accordingly, in an image forming device using a negatively-chargeable toner, the fatty acid metal salt is likely to be transferred to a non-image area on the photoconductor surface. In an image forming device using a positively-chargeable toner, the fatty acid metal salt is likely to be transferred to an image area on the photoconductor surface. Therefore, even in the case of using the negatively-chargeable toner or the positively-chargeable toner in the image forming device, the distribution of the fatty acid metal salt on the photoconductor surface is biased in the axial direction of the photoconductor. In the case where the image forming device uses the positively-chargeable toner, along with the toner particles, the fatty acid metal salt is transferred to an image-receptive media, and the amount of the fatty acid metal salt present on the photoconductor surface becomes smaller than expected. Since the fatty acid metal salt is likely to retain water in a high temperature and high humidity environment, these phenomena are more likely to be caused.

When the distribution of the fatty acid metal salt on the photoconductor surface is biased, the cleaning blade is likely to be worn especially on an area where the amount of the fatty acid metal salt is small. When the distribution of the fatty acid metal salt on the photoconductor surface is biased in the axial direction, while being in contact with the photoconductor surface, the cleaning blade strains in the axial direction; an unexpected load is applied to the cleaning blade; and the wear of the entire cleaning blade is progressed, accordingly. When the amount of the fatty acid metal salt present on the photoconductor surface is smaller than expected, the cleaning blade is more likely to be worn than expected.

For the above reasons, it is thought that when images are formed with the toner on which the fatty acid metal salt is added, the degree of wear progression of the cleaning blade is varied in the axial direction, or the progression of the wear of the entire cleaning blade is faster than expected.

Meanwhile, when the fatty acid divalent metal salt, which is the cleaning agent for toners according to the present invention, is added on a toner and used as the cleaning agent, the fatty acid divalent metal salt can impart to the toner appropriate flowability that enables exertion of excellent cleanability, and it can suppress wear of the cleaning blade even in a high temperature and high humidity environment.

The detailed mechanism is not clear; however, it is presumed as follows: the fatty acid divalent metal salt of the present invention having the water activity value A and the number average value B of the area envelopment degrees (hereinafter may be simply referred to as “area envelopment degree B”) in the ranges described above, can impart appropriate flowability to toners and is less likely to be charged by friction even in a high temperature and high humidity environment. Accordingly, when the fatty acid divalent metal salt is released from toner particles and transferred to the photoconductor surface, the distribution of the fatty acid divalent metal salt is less likely to be biased; the fatty acid divalent metal salt is uniformly distributed over the whole axial direction of the photoconductor; and the fatty acid divalent metal salt functions as a lubricant between the photoconductor and the cleaning blade, thereby suppressing wear of the cleaning blade.

The cleaning agent for toners according to the present invention is a fatty acid divalent metal salt containing 8 to 24 carbon atoms. The fatty acid divalent metal salt is a fatty acid metal salt obtained by double decomposition between a fatty acid-alkali compound salt containing 8 to 24 carbon atoms and a divalent metal salt. The fatty acid divalent metal salt containing 8 to 24 carbon atoms can be prepared by a double decomposition method in which, in an aqueous solution, a divalent metal salt is reacted with a fatty acid-alkali compound salt containing 8 to 24 carbon atoms.

The method for producing the cleaning agent for toners according to the present invention is not particularly limited. As the method, examples include, but are not limited to, a production method comprising:
preparing a slurry of a fatty acid divalent metal salt by a double decomposition method in which, in an aqueous solution, a divalent metal salt is reacted with a fatty acid-alkali compound salt obtained by reaction of a fatty acid containing 8 to 24 carbon atoms with a monovalent alkali compound,
obtaining a water-containing cake of the fatty acid divalent metal salt from the slurry, and
drying and pulverizing the water-containing cake.

The steps of the production method will be described below in detail, followed by the properties of the fatty acid divalent metal salt that is the cleaning agent for toners according to the present invention.

In the following description, the fatty acid divalent metal salt, which is the cleaning agent for toners according to the present invention, may be simply referred to as “fatty acid metal salt”.

### (Preparation of the Slurry of the Fatty Acid Metal Salt)

The slurry of the fatty acid metal salt is prepared by the double decomposition method in which, in the aqueous solution, the divalent metal salt is reacted with the fatty acid-alkali compound salt obtained by reaction of the fatty acid containing 8 to 24 carbon atoms with the monovalent alkali compound.

The fatty acid used as a raw material for the fatty acid-alkali compound salt is not particularly limited, as long as it is a fatty acid containing 8 to 24 carbon atoms. More specifically, it may be any one of a naturally-occurring fatty acid and a synthetic fatty acid, may be any one of a saturated fatty acid and an unsaturated fatty acid, and may be any one of a linear fatty acid and a branched fatty acid. In addition, a functional group such as a hydroxy group, an aldehyde group and an epoxy group may be included in the structure of the fatty acid. Preferred is a linear saturated fatty acid containing 12 to 22 carbon atoms, and more preferred is a linear saturated fatty acid containing 14 to 20 carbon atoms. When the number of the carbon atoms contained in the fatty acid is less than 8, the obtained fatty acid metal salt is not effective as a flow improver. On the other hand, when the number of the carbon atoms contained in the fatty acid is more than 24, such a fatty acid is difficult to be obtained industrially, and the solubility of the obtained fatty acid-alkali compound salt in water significantly decreases and results in poor productivity.

As the fatty acid, examples include, but are not limited to, caprylic acid, capric acid, lauric acid, myristic acid, myristoleic acid, palmitic acid, palmitoleic acid, stearic acid, oleic acid, linoleic acid, arachic acid, behenic acid, erucic acid, hydroxystearic acid and epoxystearic acid. Of them, stearic acid and myristic acid are preferred, and stearic acid is more preferred.

As the monovalent alkali compound used as a raw material for the fatty acid-alkali compound salt, examples include, but are not limited to, a hydroxide of an alkali metal (such as sodium and potassium) and an amine such as ammonia, monoethanolamine, diethanolamine and triethanolamine. From the viewpoint of obtaining high solubility in water when formed into the fatty acid-alkali compound salt, the monovalent alkali compound is preferably a hydroxide of an alkali metal such as sodium and potassium.

The fatty acid-alkali compound salt used in the present invention is obtained by reaction of the monovalent alkali compound with the fatty acid at a temperature which is generally equal to or more than the melting point of the fatty acid and at which the fatty acid is not decomposed. The temperature is preferably 100° C. or less, more preferably from 50° C. to 100° C., still more preferably from 60° C. to 90° C., and particularly preferably from 70° C. to 85° C.

As the fatty acid-alkali compound salt used in the present invention is preferably at least one selected from the group consisting of sodium stearate, potassium stearate, sodium myristate and potassium myristate, more preferably at least one selected from the group consisting of sodium stearate, potassium stearate and sodium myristate, and still more preferably at least one selected from the group consisting of sodium stearate and potassium stearate.

The fatty acid divalent metal salt, which is the cleaning agent for toners according to the present invention, is the fatty acid metal salt obtained by the double decomposition method in which, in the aqueous solution, the divalent metal salt is reacted with the fatty acid-alkali compound salt obtained above.

More specifically, the divalent metal salt is a salt of a divalent inorganic metal and an inorganic or organic acid.

As the divalent inorganic metal, examples include, but are not limited to, an alkaline-earth metal such as magnesium, calcium and barium, and a transition metal such as titanium, zinc, iron, manganese, cadmium, mercury, zirconium, lead, copper, cobalt, aluminum and nickel. Of them, preferred is at least one selected from the group consisting of zinc, calcium and magnesium, and more preferred is zinc, from the viewpoint of a light load on the environment, industrial availability, and impartation of cleanability by the obtained fatty acid metal salt.

As the divalent metal salt, examples include, but are not limited to, zinc chloride, zinc sulfate, calcium chloride, calcium acetate, magnesium chloride, magnesium sulfate and aluminum sulfate. Preferred is a chloride, sulfate or nitrate of a divalent metal such as zinc, calcium and magnesium, from the viewpoint of high solubility in water and efficient reactivity with the fatty acid-alkali compound salt.

The reaction by the above-described double decomposition method is carried out by, for example, separately preparing an aqueous solution containing the divalent metal salt and an aqueous solution containing the fatty acid-alkali compound salt and mixing them. More specifically, they are mixed by a method of adding the aqueous solution containing the divalent metal salt to the aqueous solution containing the fatty acid-alkali compound salt, a method of adding the solutions to another reaction vessel, or the like.

When mixing the solutions by, for example, gradually adding the aqueous solution containing the divalent metal salt to the aqueous solution containing the fatty acid-alkali compound salt in a dropwise manner at an appropriate speed, particles may be likely to grow and the particle diameter may be likely to increase. Accordingly, in the present invention, the fatty acid metal salt is preferably obtained by separately preparing the aqueous solution containing the fatty acid-alkali compound salt and the aqueous solution containing the divalent metal salt in different containers, putting them into another container at the same time, and reacting them. By this production method, the fatty acid metal salt having a small particle size can be obtained.

In the aqueous solution containing the fatty acid-alkali compound salt, which is used to prepare the slurry of the fatty acid metal salt, the concentration of the fatty acid-alkali compound salt is generally from 1% by mass to 10% by mass, from the viewpoint of the particle diameter of the obtained fatty acid metal salt, the productivity of the fatty acid metal salt, and the handleability of the aqueous solution containing the fatty acid-alkali compound salt or the obtained slurry of the fatty acid metal salt. The lower limit of the concentration is preferably 3% by mass or more, and more preferably 5% by mass or more. The upper limit of the concentration is preferably 8% by mass or less. When the concentration of the fatty acid-alkali compound salt is less than 1% by mass, the productivity of the fatty acid metal salt may decrease. Accordingly, the fatty acid-alkali compound salt having such a concentration is not preferable for practical use. When the concentration of the fatty acid-alkali compound salt is more than 10% by mass, the viscosity of the aqueous solution containing the fatty acid-alkali compound salt or the obtained slurry of the fatty acid metal salt increases. Accordingly, initiating a uniform reaction may be difficult.

In the aqueous solution containing the divalent metal salt, which is used to prepare the slurry of the fatty acid metal salt, the concentration of the divalent metal salt is generally from 0.1% by mass to 12% by mass, and preferably from 0.5% by mass to 10% by mass, from the viewpoint of the productivity of the fatty acid metal salt and the handleability of the aqueous solution containing the divalent metal salt or the obtained slurry of the fatty acid metal salt.

When mixing the aqueous solution containing the fatty acid-alkali compound salt and the aqueous solution containing the divalent metal salt, the amounts of the aqueous solutions are preferably adjusted so that the equivalent ratio of the divalent metal salt to the fatty acid-alkali compound salt (the divalent metal salt/the fatty acid-alkali compound salt) is from 1.80 to 2.00. When the equivalent ratio is within the range, the fatty acid metal salt in the form of particles which have a high area envelopment degree B, that is, which have less convexoconcaves, is likely to be obtained. The lower limit of the equivalent ratio is more preferably 1.85 or more, and still more preferably 1.90 or more. The upper limit of the equivalent ratio is more preferably 1.99 or less, and still more preferably 1.95 or less.

When reacting the fatty acid-alkali compound salt with the divalent metal salt, the solution temperature may be appropriately determined depending on the solubility of the fatty acid-alkali compound salt and is not particularly limited. The solution temperature is preferably from 50° C. to 100° C., and more preferably from 60° C. to 95° C. When the reaction temperature is less than 50° C., the reaction rate of the fatty acid-alkali compound salt and the divalent metal salt may decrease.

When reacting the fatty acid-alkali compound salt with the divalent metal salt, a polyalkylene glycol-based ether may be present in the aqueous solution. Accordingly, the obtained slurry of the fatty acid metal salt is likely to be stabilized, and the productivity of the fatty acid metal salt can be increased.

As the polyalkylene glycol-based ether, a triblock ether having a structure in which an oxypropylene block is sandwiched between oxyethylene blocks (EO-PO-EO) is particularly preferred.

The content of the polyalkylene glycol-based ether in the slurry of the fatty acid metal salt is generally from 0.01 parts by mass to 5 parts by mass, and preferably from 0.05 parts by mass to 2 parts by mass, with respect to 100 parts by mass of the fatty acid-alkali compound salt.

The polyalkylene glycol-based ether may be present in the reaction system before reacting the monovalent alkali compound with the fatty acid, or it may be present in the reaction system before reacting the fatty acid-alkali compound salt with the divalent metal salt.

### (Preparation of the Water-Containing Cake of the Fatty Acid Metal Salt)

The slurry of the fatty acid metal salt is obtained by the above-described method. The water-containing cake of the fatty acid metal salt is obtained from the slurry of the fatty acid metal salt. The water-containing cake of the fatty acid metal salt is obtained by, for example, filtering the slurry of the fatty acid metal salt as it is, or by separating the solvent therefrom with a centrifuge, a filter press, a vacuum rotary filtering machine or the like. As needed, before obtaining the water-containing cake of the fatty acid metal salt, washing may be carried out for removal of a byproduct inorganic salt.

The amount of water in the water-containing cake of the fatty acid metal salt is not particularly limited, and it is generally from 30% by mass to 60% by mass. The amount of the water in the water-containing cake can be measured by the same method as the amount of water in the fatty acid metal salt described below.

### (Drying and Pulverizing)

The water-containing cake of the fatty acid metal salt is dried and pulverized with an appropriate apparatus, thereby obtaining the fatty acid metal salt that is the cleaning agent for toners according to the present invention.

To obtain the fatty acid metal salt in a powdery form, flash drying at a temperature of from about 100° C. to 150° C. or tray drying has been generally employed as the drying method. Meanwhile, in the present invention, from the viewpoint of adjusting the water activity value A and area envelopment degree B of the obtained fatty acid metal salt to the predetermined values, the water-containing cake is preferably dried and pulverized by hot-air drying of the water-containing cake. By hot-air drying the water-containing cake in a high-temperature and high-speed flow, the water-containing cake can be dried and pulverized at the same time. Such a hot-air drying can be carried out by, for example, a flash dryer which is capable of instant drying in a high-temperature hot-air flow. As the flash dryer, for example, a flash jet dryer (model: FJD-4, manufactured by: Seishin Enterprise Co., Ltd.) is preferred.

In the present invention, the particle size distribution of the fatty acid metal salt is preferably adjusted by classifying the fatty acid metal salt obtained by drying and pulverizing the water-containing cake of the fatty acid metal salt. As the classifying method, a conventionally-known method can be used without any particular limitation. For example, a vibrating sieving machine for sieving by vibration can be used for classification. The fatty acid metal salt as the cleaning agent for toners according to the present invention, can be obtained in this manner.

Preferably, the drying temperature of the hot-air drying is equal to or more than the melting point of the fatty acid metal salt and is 200° C. or less. When the drying temperature is lower than the melting point of the fatty acid metal salt, the water activity value A of the obtained fatty acid metal salt may become too high. When the drying temperature is higher than 200° C., depending on the treatment time, the fatty acid metal salt may be softened, for example, and the area envelopment degree B of the fatty acid metal salt may become too low, accordingly.

The melting point of zinc stearate is 125° C.; the melting point of zinc myristate is 130° C.; the melting point of calcium stearate is 145° C.; and the melting point of magnesium stearate is 122° C.

The hot-air drying treatment time per kg of the water-containing cake of the fatty acid metal salt, is preferably 0.1 hours/kg or less, more preferably 0.08 hours/kg or less, and still more preferably 0.05 hours/kg or less. When the treatment time is longer than 0.1 hours/kg, the water activity value A of the obtained fatty acid metal salt may become too high, or the area envelopment degree B thereof may become too low. The hot-air drying treatment time is preferably 0.02 hours/kg or more. When the hot-air drying treatment time is shorter than 0.02 hours/kg, the water activity value A or area envelopment degree B of the obtained fatty acid metal salt may become too low.

### (Properties)

The fatty acid metal salt, which is the cleaning agent for toners according to the present invention, has a water activity value A of from 0.65 to 0.85 at 25° C.

The water activity value A of the fatty acid metal salt means the proportion of free water in the total amount of the water contained in the fatty acid metal salt, and it is defined as the ratio of the water vapor pressure inside the closed container containing a certain mass of the fatty acid metal salt to the vapor pressure at the temperature of the inside of the container. The water is classified into bound water, adhesional water and free water. The bound water is firmly bound to the components of the fatty acid metal salt; the adhesional water is water attached to the particle surface; and the free water refers to water present near the particles, and it is transferred or vaporized by the influence of temperature or humidity.

The water activity value A of the fatty acid metal salt at 25° C. is calculated by the following formula (1).

A=P/P₀ Formula (1)
P: The water vapor pressure (Pa) at 25° C. inside the closed container containing the fatty acid divalent metal salt
P₀: The vapor pressure (Pa) of water at 25° C.

The water activity value A can be easily measured by a conventionally-known method. For example, the water activity value A can be measured by use of a water activity meter such as AQUALAB 4TE (product name, manufactured by METER Group, Inc. (former Decagon Devices, Inc.))

In the fatty acid metal salt, which is the cleaning agent for toners according to the present invention, the water activity value A at 25° C. is only required to be from 0.65 to 0.85. From the viewpoint of further suppressing wear of the cleaning blade, the water activity value A at 25° C. is preferably from 0.69 to 0.83.

The fatty acid metal salt, which is the cleaning agent for toners according to the present invention, has a number average value B of area envelopment degrees of from 0.910 to 0.990, each of which is defined as the ratio of a projected area to an area inside an envelope (the projected area/the area inside the envelope) for a projected image, and the area envelopment degrees are those of projected images of fatty acid metal salt having an equivalent circle diameter in a range of from a cumulative 10% diameter to a cumulative 90% diameter on a volumetric basis in the fatty acid metal salt.

The “envelopment degree” is an index representing the degree of convexoconcaves on the surface of an object, and it is represented as a value in a range of from 0 to 1. The area envelopment degree is a value obtained by dividing the area of a projected image of an object by the area of a projected image of a convex shell filling a concave portion on the surface of the object, that is, by the area inside the envelope. As the convexoconcaves on the surface of the object become more complicated, the area envelopment degree value decreases. Accordingly, the object having an area envelopment degree that is lower than a certain value can be determined as an aggregated particle that is composed of attached particles.

The area envelopment degree can be measured by image analysis of the projected image of a particle with an automated image analysis system for particle size distribution measurement. For example, by using MORPHOLOGI 4, which is an automated image analysis system for particle size distribution measurement manufactured by Malvern Panalytical Ltd., in a dry dispersion system, measurement of the equivalent circle diameter, the area of a projected image and the area inside the envelope can be carried out, and the area envelopment degree can be calculated. More specifically, from the particles of a measuring population, those having an equivalent circle diameter of from a cumulative 10% diameter to a cumulative 90% diameter on a volumetric basis are extracted, and the number average value B of the area envelopment degrees of the extracted particles, each of which is defined as the ratio of the projected area to the area inside the envelope (the projected area/the area inside the envelope) for the projected image, is calculated.

For the fatty acid metal salt, which is the cleaning agent for toners according to the present invention, the number average value B of the area envelopment degrees is only required to be from 0.910 to 0.990. From the viewpoint of further suppressing wear of the cleaning blade, the number average value B of the area envelopment degrees is preferably from 0.920 to 0.990.

The volume-based median diameter (D50) of the fatty acid metal salt that is the cleaning agent for toners according to the present invention, which is measured by a laser light diffraction scattering method, is preferably 0.1 μm or more, and more preferably 0.3 μm or more. On the other hand, the median diameter (D50) is preferably 5.0 μm or less, more preferably 3.0 μm or less, and still more preferably 2.0 μm or less. when the fatty acid metal salt is aggregated, the fatty acid metal salt may be less likely to be dispersedly attached to toner particles. The fatty acid metal salt having a D50 which is equal to or more than the lower limit value, is less likely to be aggregated and is thus likely to be dispersedly attached to toner particles. When the fatty acid metal salt is coarsened, the fatty acid metal salt may be non-uniformly attached to toner particles. The fatty acid metal salt having a D50 which is equal to or less than the upper limit value has a low coarse particle presence frequency; therefore, the fatty acid metal salt is likely to be uniformly attached to toner particles.

From the viewpoint of dispersibility into toner, the amount of the water in the fatty acid metal salt, which is the cleaning agent for toners according to the present invention, is preferably 8% by mass or less, and more preferably 6% by mass or less.

The amount of the water is measured in conformity to JIS K0068 “Loss-on-Drying Method”.

### (2) Toner Composition

The toner composition of the present invention comprises the cleaning agent for toners according to the present invention and toner base particles, wherein the content of the cleaning agent for toners is from 0.01 parts by mass to 5 parts by mass, with respect to 100 parts by mass of the toner base particles.

In the toner composition of the present invention, the cleaning agent for toners according to the present invention may be added by a common toner production method. For example, the cleaning agent for toners according to the present invention may be added on conventionally-known toner particles. The toner composition of the present invention is typically a composition in which the cleaning agent of the present invention and, as needed, an external additive different from the cleaning agent are added on the surface of the toner base particles. When using the toner composition, the cleaning agent of the present invention may be released from the toner particles. In the present invention, the “toner base particles” are colored resin particles containing a binder resin, a colorant and so on, and particles in which an external additive, a cleaning agent or the like is added on a surface of the toner base particles, may be referred to as “toner particles”.

The cleaning agent of the present invention can be added on the toner base particles by a conventionally-known external addition treatment method, without any particular limitation. More specifically, for example, by adding the cleaning agent along with the external additive to the toner base particles and mixing them by a mixer or the like, the external additive and the cleaning agent can be added or fixed on the surface of the toner base particles.

As the mixer, a conventionally-known mixer can be used without any particular limitation. As the mixer, examples include, but are not limited to, a common mixer such as a turbine type mixer, a HENSCHEL MIXER and a super mixer. Of them, a HENSCHEL MIXER is preferably used.

In the toner composition of the present invention, the content (amount) of the cleaning agent for toners according to the present invention is from 0.01 parts by mass to 5 parts by mass, with respect to 100 parts by mass of the toner base particles. Since the content of the cleaning agent for toners according to the present invention is within the range, excellent flowability is provided to the toner, and the toner composition configured to suppress wear of the cleaning blade in a high temperature and high humidity environment, is provided. The content of the cleaning agent for toners according to the present invention is preferably from 0.03 parts by mass to 4 parts by mass, and more preferably from 0.05 parts by mass to 3 parts by mass, with respect to 100 parts by mass of the toner base particles.

The toner base particles contained in the toner composition of the present invention are not particularly limited. For example, the toner base particles contain a binder resin, a colorant, a charge control agent, a release agent and so on.

Examples of the binder resin, colorant, charge control agent, release agent and external additive are as shown below, and conventionally-known materials can be appropriately used.

### (Binder Resin)

As the binder resin, examples include, but are not limited to, a polyester-based resin, a styrene-(meth)acrylic acid-based copolymer resin, a thermoplastic elastomer, a styrene-based resin, a (meth)acrylic acid-based resin, an olefin-based resin (e.g., an α-olefin resin such as polyethylene and polypropylene), a vinyl-based resin (e.g., polyvinyl chloride, polyvinylidene chloride), a polyamide-based resin, a polyether-based resin, a urethane-based resin, an epoxy-based resin, a polyphenylene oxide-based resin, a terpene phenol resin, a polylactic resin, a hydrogenated rosin, a cyclized rubber and a cycloolefin copolymer resin. They can be used alone or in combination of two or more kinds. Of them, a polyester-based resin and a styrene-(meth)acrylic acid-based copolymer resin are preferred, since the demands required of toners can be satisfied with balance, such as image quality characteristics, durability and productivity.

### (Colorant)

The colorant may be a pigment or dye in black, magenta, cyan, yellow or another color.

As the black colorant, examples include, but are not limited to, a carbon black such as lamp black, thermal black, acetylene black, channel black and furnace black, and a nigrosine dye.

As the magenta colorant, examples include, but are not limited to, Rose Bengal; Dupont Oil Red; C.I. Pigment Red 1, 2, 3, 4, 5, 6, 7, 8, 9, 10, 11, 12, 13, 14, 15, 16, 17, 18, 19, 21, 22, 23, 30, 31, 32, 37, 38, 39, 40, 41, 48, 49, 50, 51, 52, 53, 54, 55, 57, 58, 60, 63, 64, 68, 81, 83, 87, 88, 89, 90, 112, 114, 122, 123, 163, 202, 206, 207 and 209; and C.I. Pigment Violet 1, 2, 10, 13, 15, 19, 23, 29 and 35.

As the cyan colorant, examples include, but are not limited to, Aniline Blue; Calco Oil Blue; Ultramarine Blue; Methylene Blue Chloride; Phthalocyanine Blue; C.I. Pigment Blue 2, 3, 15, 16 and 17; C.I. Vat Blue 6; and C.I. Acid Blue 45.

As the yellow colorant, examples include, but are not limited to, Chrome Yellow; Quinoline Yellow; and C.I. Pigment Yellow 1, 2, 3, 4, 5, 6, 7, 10, 11, 12, 13, 14, 15, 16, 17, 23, 65, 73, 74, 83, 93, 97, 128, 155 and 180.

These colorants can be used alone or in combination of two or more kinds.

As the colorant preferred for full-color use due to its good color mixing property and excellent color reproducibility, examples include, but are not limited to, C.I. Pigment Red 57 and 122 for the magenta colorant, C.I. Pigment Blue 15 for the cyan colorant, and C.I. Pigment Yellow 17, 93, 155 and 180 for the yellow colorant.

### (Charge Control Agent)

As the charge control agent, a positively- or negatively-chargeable charge control agent that is generally used to improve the chargeability of toners, can be appropriately selected and used, without any particular limitation.

As the positively-chargeable charge control agent, examples include, but are not limited to, a product modified with nigrosine; a quaternary ammonium salt such as tributylbenzylammonium-1-hydroxy-4-naphthosulfonate and tetrabutylammonium tetrafluoroborate; a diorganotin oxide such as a dibutyltin oxide, a dioctyltin oxide and a dicyclohexyltin oxide; a diorganotin borate such as a dibutyltin borate, a dioctyltin borate and a dicyclohexyltin borate; a pyridinium salt; an azine; a triphenylmethane-based compound; and a low-molecular-weight polymer containing a cationic functional group. These positively-chargeable charge control agents can be used alone or in combination of two or more kinds.

Of these positively-chargeable charge control agents, a nigrosine-based compound and a quaternary ammonium salt are preferably used.

As the negatively-chargeable charge control agent, examples include, but are not limited to, an organometallic compound such as an acetylacetone metal complex, a monoazo metal complex, a naphthoic acid-based or salicylic acid-based metal complex and their salts, a chelate compound and a low-molecular-weight polymer containing an anionic functional group. These negatively-chargeable charge control agents can be used alone or in combination of two or more kinds.

Of these negatively-chargeable charge control agents, a salicylic acid-based metal complex and a monoazo metal complex are preferably used.

The content of the charge control agent is generally from 0.1 parts by mass to 5.0 parts by mass, and preferably from 0.5 parts by mass to 3.0 parts by mass, with respect to 100 parts by mass of the binder resin.

### (Release Agent)

As the release agent, a compound that is generally used as a release agent for toners can be appropriately selected and used, without any particular limitation. As the release agent, examples include, but are not limited to, an ester wax such as stearyl stearate, stearyl behenate, behenyl stearate, behenyl behenate, stearyl montanoate, behenyl montanoate, palmityl arachidinate, pentaerythritol tetrapalmitate, pentaerythritol tetrastearate, pentaerythritol tetrabehenate, dipentaerythritol hexastearate and dipentaerythritol hexabehenate, and a hydrocarbon wax such as a polyethylene wax, a polypropylene wax, a Fischer-Tropsch wax, a paraffin wax, a microcrystalline wax and a petroleum wax.

These release agents can be used alone or in combination of two or more kinds.

### (External Additive (Flow Improver))

As needed, the toner composition of the present invention may further contain an external additive that is different from the above-described cleaning agent for toners according to the present invention. The external additive is not particularly limited, as long as it is applicable as an external additive for toners. More specifically, as the external additive, examples include, but are not limited to, metal oxide particles such as silica particles, titanium oxide particles, alumina particles, zinc oxide particles and magnetite particles, resin particles, and resin particles surface-treated with the metal oxide particles or metallic soap particles. These external additives may be used alone or in combination of two or more kinds. Of them, silica particles are preferred from the viewpoint of having excellent flowability, chargeability and abradability. The external additive may be added on the toner base particles along with the above-described cleaning agent for toners according to the present invention.

### EXAMPLES

Hereinafter, the present invention will be described in more detail, by way of examples and comparative examples.

### Examples 1 to 5 and Comparative Examples 1 to 5

### (1) Preparation of Cleaning Agents A-1 to A-5 and B-1 to B-5 for Toners

### (1-1) Preparation of a Slurry of a Fatty Acid Metal Salt

A raw material (a) that is a divalent metal salt, and a raw material (b) that is a fatty acid-alkali compound salt, both of which are shown in Table 1, were selected according to Table 2. They were each dissolved in water to prepare a component (a), which is an aqueous solution of the raw material (a), and a component (b), which is an aqueous solution of the raw material (b). The concentration of the raw material (a) in the component (a) and that of the raw material (b) in the component (b) are shown in Table 2.

Next, a slurry of a fatty acid metal salt (a fatty acid divalent metal salt) was obtained by the double decomposition method of reacting the raw material (a) with the raw material (b) in the aqueous solution by mixing the components (a) and (b) at the temperature shown in Table 2 by the following method so that the amount of the thus-obtained slurry of the fatty acid metal salt would be 500 kg.

<Method for Mixing the Component (a) and the Component (b)>

A pipeline homomixer, in which the components (a) and (b) were able to be separately supplied and mixed together by a metering pump, and a 600 L receiving container, which was equipped with a stirrer including a turbine blade (diameter 30 cm), were prepared. The turbine blade was rotated at 350 rpm. The components (a) and (b) which were in amounts that would provide the equivalent ratio (a/b) of the divalent metal salt in the component (a) to the fatty acid-alkali compound salt in the component (b) shown in Table 2 and of which temperatures were controlled to the solution temperatures shown in Table 2, were separately supplied into the pipeline homomixer, discharged therefrom and poured into the receiving container, thereby obtaining a mixed solution. The flow rates of the solutions were controlled by the metering pump so that both the sending of the component (a) solution and that of the component (b) solution would end at the same time. After the end of feeding the whole amount of the solutions, the mixed solution thus obtained was matured for 10 minutes while keeping the solution temperature, and the reaction of the mixed solution was terminated, thereby obtaining the slurry of the fatty acid metal salt.

### (1-2) Preparation of a Water-Containing Cake of the Fatty Acid Metal Salt

The obtained slurry of the fatty acid metal salt was filtered to obtain a water-containing cake of the fatty acid metal salt. The obtained cake was washed twice with water, thereby obtaining a water-containing cake of the fatty acid metal salt in which the amount of water was from 30% by mass to 60% by mass.

### (1-3) Drying and Pulverizing

Using a flash jet dryer (model: FJD-4) manufactured by Seishin Enterprise Co., Ltd., the water-containing cake of the fatty acid metal salt obtained above was subjected to hot-air drying in the drying condition shown in Table 2 (flow temperature, treatment time per kg of the water-containing cake of the fatty acid metal salt) to dry and pulverize the water-containing cake. In addition, the pulverized water-containing cake was classified by a vibrating sieving machine having an opening size of 760 μm, thereby obtaining a fatty acid metal salt that was the cleaning agent for toners.

TABLE 1

| | | |
|---|---|---|
| Raw material (a) | (a-1) | Zinc sulfate |
| | (a-2) | Zinc chloride |
| | (a-3) | Calcium chloride |
| | (a-4) | Magnesium sulfate |
| Raw material (b) | (b-1) | Sodium stearate |
| | (b-2) | Potassium stearate |
| | (b-3) | Sodium myristate |

TABLE 2

| | | Synthesis condition | | | | | | | |
|---|---|---|---|---|---|---|---|---|---|
| | | | Solid | | Solid | | | | |
| | | | component | | component | | | Flash jet dryer drying | |
| | | | concentration | | concentration | | | condition | |
| | | Raw | a (%) in | Raw | b (%) in | | Solution | Flow | Treatment |
| | | material | aqueous | material | aqueous | Equivalent | temperature | temperature | time |
| Cleaning agent | | (a) | solution | (b) | solution | ratio (a/b) | (° C.) | (° C.) | (h/kg) |
| Example 1 | A-1 | (a-1) | 1.9 | (b-1) | 7 | 1.90 | 80 | 190 | 0.04 |
| Example 2 | A-2 | (a-2) | 1.5 | (b-2) | 7 | 1.95 | 60 | 170 | 0.04 |
| Example 3 | A-3 | (a-1) | 3.2 | (b-3) | 9.5 | 1.85 | 80 | 150 | 0.02 |
| Example 4 | A-4 | (a-3) | 1.1 | (b-1) | 6 | 1.85 | 80 | 150 | 0.03 |
| Example 5 | A-5 | (a-4) | 1.5 | (b-1) | 7 | 1.90 | 60 | 160 | 0.08 |
| Comparative | B-1 | (a-1) | 1.9 | (b-1) | 7 | 1.80 | 80 | 170 | 0.01 |
| Example 1 | | | | | | | | | |
| Comparative | B-2 | (a-1) | 1.8 | (b-2) | 7 | 1.85 | 80 | 150 | 12 |
| Example 2 | | | | | | | | | |
| Comparative | B-3 | (a-3) | 1.1 | (b-1) | 6 | 1.90 | 80 | 230 | 0.01 |
| Example 3 | | | | | | | | | |
| Comparative | B-4 | (a-4) | 1.5 | (b-1) | 7 | 1.95 | 60 | 60 | 0.55 |
| Example 4 | | | | | | | | | |
| Comparative | B-5 | (a-1) | 1.9 | (b-1) | 7 | 1.90 | 80 | 210 | 0.05 |
| Example 5 | | | | | | | | | |

The fatty acid metal salts A-1 to A-5 and B-1 to B-5 (the cleaning agents for toners) obtained in the examples and the comparative examples were measured as follows. The measurement results are shown in Table 3.

### (Water Activity Value A)

The fatty acid metal salt was put in a constant-temperature closed container at 25° C. The water activity value A calculated by the following formula (1) using the water vapor pressure in the container when the water on the particle surface was at equilibrium with the water of the surrounding air and the water vapor pressure of pure water in the same condition, was measured.

$$A = P / {P}_{0} \qquad Formula ( 1 )$$

P: Water vapor pressure (Pa) at 25° C. inside the closed container containing the fatty acid divalent metal salt
P₀: Vapor pressure (Pa) of water at 25° C.

More specifically, 10 g of the fatty acid metal salt was put in a sample cup (outer diameter 40 mm, inner diameter 39.4 mm, height 11.4 mm, capacity 15 mL, a cap made of a high-density polyethylene). The sample cup was set in the chamber of AQUALAB 4TE (manufactured by METER Group, Inc. (former Decagon Devices, Inc.)) to measure the water activity value A at 25° C. calculated by the formula (1). The relative humidity in the room when measuring the water activity value A was 50% RH.

### (Number Average Value B of the Area Envelopment Degrees)

As a measurement sample, 3 mm³ of fatty acid metal salt was used. Using MORPHOLOGI 4 (an automated image analysis system for particle size distribution measurement manufactured by Malvern Panalytical Ltd.) equipped with ×10 and ×50 objective lenses, measurement of the fatty acid metal salt in a dry dispersion system was carried out. From the particles of the measuring population, those having an equivalent circle diameter in a range of from a cumulative 10% diameter to a cumulative 90% diameter on a volumetric basis were extracted, and the area envelopment degree of each of the extracted particles was obtained. The area envelopment degree was defined as the ratio of the projected area to the area inside the envelope (the projected area/the area inside the envelope). The number average value was calculated from the area envelopment degrees of the extracted particles, thereby calculating the number average value B of the area envelopment degrees.

### (Amount of Water (% by Mass))

The amount of water remaining in the fatty acid metal salt was measured in conformity to JIS K0068 “Loss-on-drying method”. More specifically, 3 g of the fatty acid metal salt was weighed out in a precisely weighed weighing bottle; the weighing bottle was precisely weighed again; the weighing bottle was left to stand for three hours to dry in a dryer at 105° C.±2° C.; the weighing bottle was cooled down for 60 minutes in a desiccator and then precisely weighed again; and then the proportion of the lost weight was calculated as the amount of the water (% by mass).

### (Median Diameter (D50) (μm))

To 0.05 g of fatty acid metal salt, 40 ml of purified water and 0.001 g of polyoxyethylene nonylphenyl ether (NONION NS-210 manufactured by NOF Corporation) were added. They were dispersed for 10 minutes by use of an ultrasonic disperser, thereby preparing a measurement dispersion.

Next, the obtained measurement dispersion was put in laser diffraction particle size analyzer MICROTRAC MT3200II manufactured by Nikkiso Co., Ltd., in which water was circulated as a circulating fluid, to measure the particle size distribution of the fatty acid metal salt. From the measured values obtained as volume distribution, the particle diameter at 50% cumulative particle size distribution on a volumetric basis was calculated as the median diameter (D50). In the examples and the comparative examples, measurement was carried out by use of laser diffraction particle size analyzer MICROTRAC MT3200II manufactured by Nikkiso Co., Ltd. Also, a measurement device having the same function as MT3200II may be used.

TABLE 3

| | | Analysis values | | | | |
|---|---|---|---|---|---|---|
| | | Water | Area | Amount of | | Melting |
| | | activity | envelopment | water (% | D50 | point |
| Cleaning agent | | value A | degree B | by mass) | (μm) | (° C.) |
| Example 1 | A-1 | 0.74 | 0.983 | 0.10 | 0.5 | 125 |
| Example 2 | A-2 | 0.82 | 0.965 | 0.14 | 0.3 | 125 |
| Example 3 | A-3 | 0.71 | 0.935 | 0.05 | 1.0 | 130 |
| Example 4 | A-4 | 0.68 | 0.972 | 2.73 | 1.3 | 145 |
| Example 5 | A-5 | 0.85 | 0.918 | 5.05 | 1.9 | 122 |
| Comparative | B-1 | 0.58 | 0.974 | 0.03 | 0.5 | 125 |
| Example 1 | | | | | | |
| Comparative | B-2 | 0.89 | 0.963 | 0.09 | 0.4 | 125 |
| Example 2 | | | | | | |
| Comparative | B-3 | 0.38 | 0.895 | 2.80 | 1.3 | 145 |
| Example 3 | | | | | | |
| Comparative | B-4 | 1.15 | 0.983 | 4.95 | 1.8 | 122 |
| Example 4 | | | | | | |
| Comparative | B-5 | 0.66 | 0.904 | 0.04 | 0.7 | 125 |
| Example 5 | | | | | | |

### (2) Preparation of Toner Compositions C-1 to C-10

First, 90.5 parts by mass of a polyester resin (product name: DIACRON ER-508, manufactured by: Mitsubishi Chemical Corporation), 4.5 parts by mass of a behenyl behenate (product name: UNISTER M-2222SL, manufactured by NOF Corporation) and 4.5 parts by mass of Pigment Yellow 180 (product name: TONER YELLOW HG, manufactured by: Clariant Corporation) were mixed by a HENSCHEL MIXER to obtain a mixture. Using a biaxial kneading extruder, the mixture was melt-kneaded at 130° C. to obtain a kneaded product. The kneaded product was gradually cooled down to room temperature. Then, the cooled product was coarsely pulverized by a cutter mill. The coarsely pulverized product was further pulverized by a fine pulverizer using a jet flow. In addition, the pulverized product was classified by use of an air classifier, thereby obtaining toner base particles which were yellow colored particles.

To 100 parts by mass of the toner base particles, 2.5 parts by mass of hydrophobic silica particles (RY50 manufactured by Nippon Aerosil Co., Ltd.) and, among the cleaning agents A-1 to A-5 and B-1 to B-5, 0.1 parts by mass of the cleaning agent for toners shown in Table 4 were added at the same time. They were mixed by a HENSCHEL MIXER at a peripheral speed of 30 m/see for three minutes. Then, the obtained mixture was classified by a vibrating sieving machine having an opening size of 45 μm, thereby obtaining a toner composition (an external addition toner).

### (Flowability Evaluation)

Based on the angle of difference of the toner composition, which was measured by powder characteristics tester PT-X manufactured by Hosokawa Micron Corporation, the flowability of the toner composition was evaluated.

The angle of difference of the toner composition was measured as follows, based on the method proposed by Carr, which is described in Carr, R. L., Chem. Eng., Jan. 18, 1965, Vol. 72.

As the measuring device, powder characteristics tester PT-X (manufactured by Hosokawa Micron Corporation) equipped with a digital vibration meter was used.

After passing through a 710 μm sieve, the toner composition was freely fallen from a certain height to a circular specimen mount, thereby shaping the toner composition in a corn form. The angle of the hypotenuse of the cone was measured as an angle of repose.

Next, the circular specimen mount was tapped up and down three times in a certain condition, thereby collapsing the cone of the toner composition. The angle of the hypotenuse of the collapsed cone was measured as an angle of collapse.

Using the angle of repose and the angle of collapse, an angle of difference was calculated by the following formula.

Angle of difference (°)=Angle of repose (°)−Angle of collapse (°)

The angle of collapse is an angle between, when collapsing a part of the cone used for the repose angle measurement by making a specified impact on the cone, the generatrix of the cone and the horizontal plane thereof. As the angle of collapse decreases, the powder is likely to naturally flow. When the cone does not collapse despite the specific impact made thereon, the angle of repose is the same angle as the angle of collapse. The angle of difference is the angle of difference between the angle of repose and the angle of collapse. As the angle of difference increases, the floodability of the powder increases. When the angle of repose is the same angle as the angle of collapse, the angle of difference is 0°.

For the toner compositions C-1 to C-5 containing the cleaning agents A-1 to A-5, respectively, the angle of difference between the angle of repose and the angle of collapse was from 17° to 19°. Accordingly, the toner compositions C-1 to C-5 had excellent cleanability and had no problems such as toner scatter. Especially, the toner compositions C-1 and C-2, each of which had an angle of difference in a range of from 18° to 19°, were particularly excellent in cleanability. When the angle of difference is more than 19°, the floodability of the toner is too high, and problems such as toner scatter is caused. When the angle of difference is less than 17°, the flowability of the toner remarkably deteriorates and results in poor cleanability. Therefore, based on the angle of difference, the flowability of the toner compositions was evaluated as follows.

### <Flowability Evaluation Criteria>

A: The angle of difference is 18° or more and 19° or less.

B: The angle of difference is 17° or more and less than 18°.

F: The angle of difference is less than 17° or more than 19°.

### (Cross-Sectional Wear Area in High Temperature and High Humidity Environment)

A commercially-available color printer was prepared. The toner composition (negatively-chargeable) was loaded in the developing device of the printer. With this image forming device, an image was printed on 1000 sheets at an image density of 5% in a high temperature and high humidity environment (temperature 30° C., relative humidity 85%).

In this image formation, the center in the axial direction of the photoconductor was an image area, and a position 10 cm away from the center in the axial direction of the photoconductor was a non-image area.

Before and after the image formation, the cross-sectional profile of the cleaning blade of the photoconductor was observed with a laser microscope (VK-9500 manufactured by Keyence Corporation). A region having an axial direction length of 80 μm was observed at the following two positions of the cross-sectional profile: the center in the axial direction of the cleaning blade (an image area) and a position 10 cm away from the center in the axial direction (a non-image area). For each of the two positions, the difference in the cross-sectional area before and after the image formation was calculated and defined as the cross-sectional wear area (μm²). The “axial direction of the cleaning blade” was the same direction as the axial direction of the photoconductor.

As the cross-sectional wear area decreases, the effect of suppressing wear of the cleaning blade increases. Accordingly, based on the cross-sectional wear area, the effect of suppressing wear of the cleaning blade was evaluated as follows.

### <Wear Suppressing Effect Evaluation Criteria>

A: The cross-sectional wear area is 20 μm² or less.

B: The cross-sectional wear area is more than 20 μm² and 40 μm² or less.

F: The cross-sectional wear area is more than 40 μm².

TABLE 4

| | | Cleaning agent | | Angle of | Angle of | Angle of | |
|---|---|---|---|---|---|---|---|
| Toner | | | Parts by | repose | collapse | difference | Cross-sectional |
| composition | | Type | mass | (°) | (°) | (°) | wear area (μm²) |
| Example 1 | C-1 | A-1 | 0.1 | 53.1 | 34.3 | 18.8 (A) | 18 (A) |
| Example 2 | C-2 | A-2 | 0.1 | 52.9 | 34.8 | 18.1 (A) | 15 (A) |
| Example 3 | C-3 | A-3 | 0.1 | 53.7 | 36.2 | 17.5 (B) | 18 (A) |
| Example 4 | C-4 | A-4 | 0.1 | 52.9 | 35.3 | 17.6 (B) | 29 (B) |
| Example 5 | C-5 | A-5 | 0.1 | 52.5 | 35.1 | 17.4 (B) | 38 (B) |
| Comparative | C-6 | B-1 | 0.1 | 52.1 | 35.3 | 16.8 (F) | 56 (F) |
| Example 1 | | | | | | | |
| Comparative | C-7 | B-2 | 0.1 | 53.4 | 36.9 | 16.5 (F) | 62 (F) |
| Example 2 | | | | | | | |
| Comparative | C-8 | B-3 | 0.1 | 51.4 | 31.8 | 19.6 (F) | 46 (F) |
| Example 3 | | | | | | | |
| Comparative | C-9 | B-4 | 0.1 | 52.8 | 36.9 | 15.9 (F) | 91 (F) |
| Example 4 | | | | | | | |
| Comparative | C-10 | B-5 | 0.1 | 52.9 | 36.8 | 16.1 (F) | 39 (B) |
| Example 5 | | | | | | | |

The toner compositions C-1 to C-5 prepared in Examples 1 to 5 contained the cleaning agents A-1 to A-5 for toners, respectively. Each of the cleaning agents A-1 to A-5 was a fatty acid divalent metal salt obtained by double decomposition between a fatty acid-alkali compound salt containing 8 to 24 carbon atoms and a divalent metal salt, and it had a water activity value A of from 0.65 to 0.85 and an area envelopment degree B of from 0.910 to 0.990. Accordingly, the toner compositions C-1 to C-5 had no problems such as toner scatter; they had appropriate flowability that enables exertion of excellent cleanability; and they were less likely to cause wear of the cleaning blade in a high temperature and high humidity environment. Of them, the toner compositions C-1 to C-3 of Examples 1 to 3 were especially less likely to cause wear of the cleaning blade in a high temperature and high humidity environment. This is presumed to be because, in the cleaning agents A-1 to A-3 contained in the toner compositions C-1 to C-3, the water activity value A was in a more preferred range of from 0.69 to 0.83, and the area envelopment degree B was in a more preferred range of from 0.920 to 0.990. In addition, the toner compositions C-1 and C-2 of Examples 1 and 2 were particularly excellent in flowability. This is presumed to be because the cleaning agents A-1 and A-2 contained in the toner compositions C-1 and C-2 had an excellent balance between the water activity value A and the area envelopment degree B.

In Examples 1 to 5, for the preparation of the fatty acid metal salt (the cleaning agent for toners), the fatty acid-alkali compound salt containing 8 to 24 carbon atoms and the divalent metal salt were used as raw materials, and the drying condition of hot-air drying of the water-containing cake of the fatty acid metal salt, which was obtained from the slurry, at high temperature was controlled, thereby controlling the water activity value A to 0.65 to 0.85 and the area envelopment degree B to 0.910 to 0.990 for the obtained fatty acid metal salt. In a conventional production of a fatty acid metal salt which is used as a cleaning agent or external additive for toners, drying is generally carried out in the condition similar to the condition in Comparative Example 2.

Meanwhile, the toner composition C-6 prepared in Comparative Example 1 had too low flowability and thus poor cleanability, and it was likely to cause wear of the cleaning blade in a high temperature and high humidity environment because, for the cleaning agent B-1 used in the toner composition C-6, the water activity value A was less than 0.65 even though the area envelopment degree B was in a range of from 0.910 to 0.990.

The toner compositions C-7 and C-9 prepared in Comparative Examples 2 and 4, respectively, had too low flowability and thus poor cleanability, and they were likely to cause wear of the cleaning blade in a high temperature and high humidity environment because, for the cleaning agents B-2 and B-4 used in the toner compositions C-7 and C-9, respectively, the water activity value A was more than 0.85 even though the area envelopment degree B was in a range of from 0.910 to 0.990.

The toner composition C-8 prepared in Comparative Example 3 had too high floodability, and it was likely to cause problems such as toner scatter and to cause wear of the cleaning blade in a high temperature and high humidity environment because, for the cleaning agent B-3 used in the toner composition C-8, the water activity value A was less than 0.65, and the area envelopment degree B was less than 0.910.

The toner composition C-10 prepared in Comparative Example 5 had too low flowability and thus poor cleanability because, for the cleaning agent B-5 used in the toner composition C-10, the area envelopment degree B was less than 0.910 even though the water activity value A was in a range of from 0.65 to 0.85.

1. A cleaning agent for toners, which is a fatty acid divalent metal salt obtained by double decomposition between a fatty acid-alkali compound salt, which is obtained by reaction of a fatty acid containing 8 to 24 carbon atoms with a monovalent alkali compound, and a divalent metal salt,
wherein the fatty acid divalent metal salt is in a form of particles,
wherein the fatty acid divalent metal salt has a water activity value A of from 0.65 to 0.85 at 25° C., which is calculated by the following formula (1),
$$A = P / {P}_{0} \qquad Formula ( 1 )$$
where P is water vapor pressure (Pa) at 25° C. inside a closed container containing the fatty acid divalent metal salt, and P₀ is vapor pressure (Pa) of water at 25° C., and
wherein the fatty acid divalent metal salt has a number average value B of area envelopment degrees of from 0.910 to 0.990, each of which is defined as a ratio of a projected area to an area inside an envelope (the projected area/the area inside the envelope) for a projected image, and the area envelopment degrees are those of projected images of particles having an equivalent circle diameter in a range of from a cumulative 10% diameter to a cumulative 90% diameter on a volumetric basis in all of the particles of the fatty acid divalent metal salt.

2. The cleaning agent for toners according to claim 1, wherein a volume-based median diameter (D50) of the fatty acid divalent metal salt in the form of particles, which is measured by a laser light diffraction scattering method, is from 0.3 μm to 5.0 μm.

3. The cleaning agent for toners according to claim 1, wherein a divalent metal contained in the fatty acid divalent metal salt is at least one selected from the group consisting of zinc, calcium and magnesium.

4. A method for producing the fatty acid divalent metal salt, which is the cleaning agent for toners defined by claim 1,
the method comprising:
preparing a slurry of a fatty acid divalent metal salt by a double decomposition method in which, in an aqueous solution, a divalent metal salt is reacted with a fatty acid-alkali compound salt obtained by reaction of a fatty acid containing 8 to 24 carbon atoms with a monovalent alkali compound,
obtaining a water-containing cake of the fatty acid divalent metal salt from the slurry, and
drying and pulverizing the water-containing cake by hot-air drying of the water-containing cake at a temperature which is equal to or more than a melting point of the fatty acid divalent metal salt and is 200° C. or less.

5. A toner composition comprising the cleaning agent for toners defined by claim 1 and toner base particles,
wherein a content of the cleaning agent for toners is from 0.01 parts by mass to 5 parts by mass, with respect to 100 parts by mass of the toner base particles.
